(19) United States
(12) Patent Application Publication
(10) Pub. No.: US 2025/0192286 A1
(43) Pub. Date: Jun. 12, 2025

(54) BATTERY PACK HAVING THERMAL RADIATION CONTROL SURFACE TREATMENT

(71) Applicant: Medtronic, Inc., Minneapolis, MN (US)

(72) Inventors: Laura E. McCalla, Brossard (CA); Parthasarathy M. Gomadam, Plymouth, MN (US); Erik J. Hovland, Minnetonka, MN (US); Marissa A. Carey, St. Paul, MN (US); Eric I. Hanson, Maple Grove, MN (US)

(21) Appl. No.: 18/976,422

(22) Filed: Dec. 11, 2024

Related U.S. Application Data
(60) Provisional application No. 63/608,972, filed on Dec. 12, 2023.

(51) Int. Cl. H01M 10/658; A61B 17/00; H01M 10/0525; H01M 10/623; H01M 50/24; H01M 50/247

(52) U.S. Cl. CPC H01M 10/658; A61B 17/00; H01M 10/0525; H01M 10/623; H01M 50/24; H01M 50/247; A61B 2017/00734; H01M 2220/30

(57) ABSTRACT

Battery packs including electrochemical cells and having a thermal radiation control surface treatment to modify radiative transfer of thermal energy into and from the electrochemical cell are provided herein. More specifically, battery packs including a pack enclosure, an electrochemical cell disposed within the pack enclosure, and a thermal radiation control surface treatment on an exterior surface of the electrochemical cell, an interior surface of the pack enclosure, or both, are provided herein.

### RELATED APPLICATIONS

This application claims the benefit of U.S. Provisional Application Ser. No. 63/608,972, filed Dec. 12, 2023, the entire contents of each of which are incorporated herein by reference.

### TECHNICAL FIELD

The present disclosure generally relates to the field of lithium-ion batteries or cells.

### BACKGROUND

For many surgical powered tools, the ability to be both cordless and rechargeable is generally beneficial, and may help improve ease-of-use and patient outcomes. For such high-powered, high-energy applications, the use of lithium-ion battery technology is particularly well suited. In order to be used in an operating room, however, the surgical instrument must be sterilized.

In general, there are three approaches used to address this problem, including, for example, aseptic transfer of the battery into a sterile device, which typically requires two people: a first person to handle the unsterile battery and a second person to remain in the sterile field and only handle the sterile (e.g., autoclaved) device after the battery is inserted by the first person. Another typical approach is STERRAD, which uses hydrogen peroxide vapor and low-temperature gas plasma low temperature sterilization of the battery pack (with ambient temperatures typically less than 55° C.) and requires specialized additional equipment. A third typical approach is aseptic transfer into sterile clamshell, which requires additional packaging and two people for aseptic transfer, as with the aseptic transfer into sterile device approach. In order to improve ease-of-use and reduce or avoid the need for additional specialized hospital equipment, it would be beneficial to be able to autoclave the entire instrument, including the battery. In order to autoclave the entire instrument, the lithium-ion battery must withstand a standard steam autoclave cycle (e.g., 134° C. for 18 minutes) and maintain usability at application temperature for a desirable number of charge-discharge cycles, such as for 100 to 300 cycles.

Many components of current commercial lithium-ion batteries cannot withstand the extreme temperatures of high-temperature autoclave, such as steam autoclave. For example, polyethylene, a component of a commonly used tri-layer shutdown separator, melts at or below 130° C. As another example, linear carbonate solvents used in commercial lithium-ion batteries often have a low boiling point (e.g., less than 140° C.). As yet another example, LiPF₆, a commonly used electrolyte salt, decomposes near 80° C. Furthermore, corrosion of a battery's metal current collectors is greatly accelerated at high temperatures, which can lead to delamination of the electrode active material.

Generally, under high-temperature conditions, heat stress on a standard battery may affect battery performance, such as reducing the number of charge-discharge cycles of which it is capable. Two key battery attributes for rechargeable batteries are capacity retention and discharge rate retention. Capacity retention may be described as measuring a battery's specific capacity over the course of multiple charge-discharge cycles as a percentage of the battery's specific capacity in the battery's first cycle. Similarly, discharge rate retention may be described as measuring a battery's discharge rate (that is, the time rate of energy transfer of which the battery is capable) over the course of multiple charge-discharge cycles as a percentage of the battery's discharge rate in the battery's first cycle. Heat stress on a standard battery may reduce discharge rate retention, and/or reduce capacity retention.

Thus, for at least the reasons described herein, lithium-ion batteries capable of withstanding the conditions of steam autoclave conditions are needed.

### SUMMARY

The present disclosure is directed to lithium-ion batteries or cells. More specifically, the present disclosure is directed to lithium-ion batteries and battery packs having thermal radiation control coatings to reduce transfer of thermal energy, or heat, via radiation from an outside environment, such as to reduce transfer of thermal energy via radiation to the lithium-ion batteries during a high-temperature autoclave operation. Without wishing to be bound by theory, reducing radiative heat transfer to the lithium-ion cell, or cells, may reduce stress on the cell during high-temperature autoclave and thereby improve the number of charge-discharge cycles of which the cell is capable. In other words, the battery packs with thermal radiation control surface treatments, as described herein, may have improved rate retention, improved capacity retention, or a combination thereof.

Embodiments disclosed herein may include a battery pack including a pack enclosure having a pack interior surface, an electrochemical cell disposed within the pack enclosure having a cell exterior surface facing the pack interior surface, and a cell thermal radiation control surface treatment on at least a portion of the cell exterior surface and disposed between the pack interior surface and the cell exterior surface. The pack interior surface may have a pack surface radiative emissivity value and a pack surface absorptive emissivity value, the cell exterior surface may have a cell surface radiative emissivity value and a cell surface absorptive emissivity value, and the cell surface treatment may have a cell surface treatment absorptive emissivity value at a temperature of 100° C. or greater that is less than the cell surface absorptive emissivity value at the temperature.

The cell thermal radiation control surface treatment may include a thermochromic compound. The cell surface treatment absorptive emissivity value at the temperature may be less than the cell surface treatment absorptive emissivity value at room temperature, the cell surface treatment radiative emissivity value at the temperature may be greater than the cell surface treatment radiative emissivity value at room temperature, or both. The cell surface treatment absorptive emissivity value at room temperature may be greater than the cell surface absorptive emissivity value at room temperature, the cell surface treatment radiative emissivity value at room temperature may be greater than the cell surface radiative emissivity value at room temperature, or both. The cell surface treatment absorptive emissivity value at the temperature may be 0.4 or less, 0.3 or less, 0.2 or less, 0.1 or less, or between 0 and 0.5. The cell surface treatment radiative emissivity value at the temperature may be 0.6 or greater, 0.7 or greater, 0.8 or greater, 0.9 or greater, or between 0.6 and 1. The cell thermal radiation control surface treatment may include a reflective surface.

The battery pack may further include a pack thermal radiation control surface treatment on at least a portion of the pack interior surface and disposed between the pack interior surface and the cell exterior surface. The pack thermal radiation surface treatment may have a pack surface treatment absorptive emissivity value greater than the pack surface absorptive emissivity value, a pack surface treatment radiative emissivity value greater than the pack surface radiative emissivity value, or both. The pack thermal radiation control surface treatment may have a pack surface treatment absorptive emissivity value less than the pack surface absorptive emissivity value, a pack surface treatment radiative emissivity value less than the pack surface radiative emissivity value, or both. The pack thermal radiation control surface treatment may include a surface roughness to increase radiative emissivity of the pack interior surface, increase absorptive emissivity of the pack interior surface, or both.

Further embodiments disclosed herein may include a battery pack including a pack enclosure with a pack interior surface, an electrochemical cell disposed within the pack enclosure and having a cell exterior surface facing the pack interior surface, and a pack thermal radiation control surface treatment on at least a portion of the pack interior surface and disposed between the pack interior surface and the cell exterior surface. The pack interior surface may have a pack surface radiative emissivity value and a pack surface absorptive emissivity value. The cell exterior surface may have a cell surface radiative emissivity value and a cell surface absorptive emissivity value. The pack surface treatment may have a pack surface treatment absorptive emissivity value at a temperature of 100° C. or greater that is greater than the pack surface absorptive emissivity value at the temperature, a pack surface treatment radiative emissivity value at the temperature that is greater than the pack surface radiative emissivity value at the temperature, or both.

The pack surface treatment absorptive emissivity value at room temperature may be greater than the pack surface absorptive emissivity value at room temperature; the pack surface treatment radiative emissivity value at room temperature may be greater than the pack surface radiative emissivity value at room temperature, or both. The pack surface treatment absorptive emissivity value at the temperature may be 0.6 or greater, 0.7 or greater, 0.8 or greater, 0.9 or greater, or between 0.6 and 1. The pack surface treatment radiative emissivity value at the temperature may be 0.6 or greater, 0.7 or greater, 0.8 or greater, 0.9 or greater, or between 0.6 and 1. The battery pack may further include a cell thermal radiation control surface treatment on at least a portion of the cell exterior surface and disposed between the pack interior surface and the cell exterior surface. The cell thermal radiation control surface treatment may have a cell surface treatment absorptive emissivity value less than the cell surface absorptive emissivity value, a cell surface treatment radiative emissivity value greater than the cell surface radiative emissivity value, or both.

Still further embodiments disclosed herein may include a battery pack having a pack enclosure with a pack interior surface, an electrochemical cell disposed within the pack enclosure and having a cell exterior surface facing the pack interior surface, and a cell thermal radiation control surface treatment on at least a portion of the cell exterior surface and disposed between the pack interior surface and the cell exterior surface. The cell thermal radiation control surface treatment may have a cell coating absorptive emissivity value of 0.4 or less, 0.3 or less, 0.2 or less, or 0.1 or less at a temperature of 100° C. or greater.

The cell thermal radiation control surface treatment may include a thermochromic compound. The cell surface treatment absorptive emissivity value at the temperature of 100° C. or greater may be less than the cell surface treatment absorptive emissivity value at room temperature, the cell surface treatment radiative emissivity value at the temperature may be greater than the cell surface treatment radiative emissivity value at room temperature, or both. The cell surface treatment absorptive emissivity value at room temperature may be greater than the cell surface absorptive emissivity value at room temperature; the cell surface treatment radiative emissivity value at room temperature may be greater than the cell surface radiative emissivity value at room temperature, or both. The cell surface treatment absorptive emissivity value at the temperature of 100° C. or greater may be less than the cell surface absorptive emissivity value at the temperature, the cell surface treatment radiative emissivity value at the temperature may be greater than the cell surface radiative emissivity at the temperature, or both.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a cross section side view of an illustrative electrochemical cell.

FIG. 1B is an isometric view of the electrochemical cell of FIG. 1A with a cell housing.

FIG. 2 is a cross-sectional representation of an illustrative battery pack including the electrochemical cell of FIG. 1A and having a cell thermal radiation control surface treatment.

FIG. 3 is a cross-sectional representation of the illustrative battery pack of FIG. 2 having a pack thermal radiation control surface treatment.

FIG. 4 is a cross-sectional representation of the illustrative battery pack of FIG. 2 having both a cell thermal radiation control surface treatment and a pack thermal radiation control surface treatment.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various illustrative embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art, unless otherwise specified. The definitions provided herein are to facilitate understanding of certain terms used frequently herein and are not meant to limit the scope of the present disclosure.

Unless otherwise indicated, the terms “polymer,” “polymerized monomers,” and “polymeric material” include, but are not limited to, organic homopolymers, copolymers, such as, for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof”′ and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. and 10 or less includes 10, 9.4, 7.6, 5, 4.3, 2.9, 1.62, 0.3, etc.). Where a range of values is “up to,” “at most,” or “at least” a particular value, that value is included within the range.

As used herein, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Such inclusive or open-ended words encompass more restrictive or closed terms or phrases, such as “consisting” or “consisting essentially.”

Reference in the specification to “some embodiments,” “an embodiment,” “one embodiment,” “at least one embodiment,” “embodiments,” “one or more embodiments,” or “other embodiments” means that a particular feature, structure, aspect, or characteristic described in connection with the embodiments is included in at least some embodiments, but not necessarily all embodiments, of the present disclosures.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the present disclosure.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

A typical battery includes one or more cells that includes a negative electrode (e.g., an anode), a positive electrode (e.g., a cathode), a separator between the negative and positive electrodes; and an electrolyte contacting the negative electrode, positive electrode, and the separator.

Such batteries can be used in a wide variety of surgical powered tools, devices, and instruments, including, for example, ultrasonic dissectors, vessel sealing devices, staplers, orthopedic saws/drills, or the modular surgical device disclosed in U.S. Pat. No. 9,017,355 (Smith et al.). More generally, the types of surgical devices include hand-held, powered medical diagnostic and surgical tools and wearable medical devices. Examples include RF-powered (radio frequency-powered) surgical sealing devices, nerve integrity monitoring devices, ablation devices, powered atherectomy devices, external, wearable stimulation devices, and external, wearable diagnostic devices.

Typical lithium-ion batteries operate within a narrow temperature range, for example from −20° C. to +60° C., with a high delivered power at room temperature. When batteries are exposed to high temperature external environments, thermal energy generally transfers from the outside environment through the outer case via conduction. The thermal energy generally transfers from the outer case, or pack enclosure to battery cells inside the outer case via three mechanisms: conduction, convection, and radiation.

Thermal energy transfer by radiation may be described as the transfer of thermal energy, or heat, by emission and absorption of electromagnetic radiation generated by the thermal motion of particles in matter. In general, thermal radiation may transfer energy from a first object to a second object by emission of electromagnetic radiation from the surface of the first object and absorption of the electromagnetic radiation by the surface of the second object. Likewise, thermal radiation may transfer energy from the second object to the first object by emission of electromagnetic radiation from the surface of the second object and absorption of the electromagnetic radiation by the surface of the first object.

The amount of thermal energy transferred is affected by the emissivity of each surface. Emissivity of a surface may be defined by a combination of absorptive emissivity and radiative emissivity. In other words, a surface may be described as having a radiative emissivity value and an absorptive emissivity value. In general, the radiative emissivity value of a surface may characterize how well the surface radiates thermal energy, or transfers away the thermal energy. Conversely, the absorptive emissivity value of a surface may characterize how well the surface absorbs radiated thermal energy, or takes in radiated thermal energy.

Emissivity may be measured, for example, using a thermal radiation detector (e.g., a thermopile or a bolometer) to compare the measured thermal radiation from a subject surface with the measured thermal radiation from a nearly ideal, black sample.

As described herein, thermal energy transfer to the interior cells of a battery pack, for example during high-temperature autoclave (e.g., steam autoclave), may be reduced by limiting transfer of thermal energy via radiation. As further described herein, transfer of thermal energy via radiation may be limited using surface treatments, such as surface coatings or other modifications to the surface, to selectively modify the emissivity of exterior surfaces of the cell, interior surfaces of the outer case, or both.

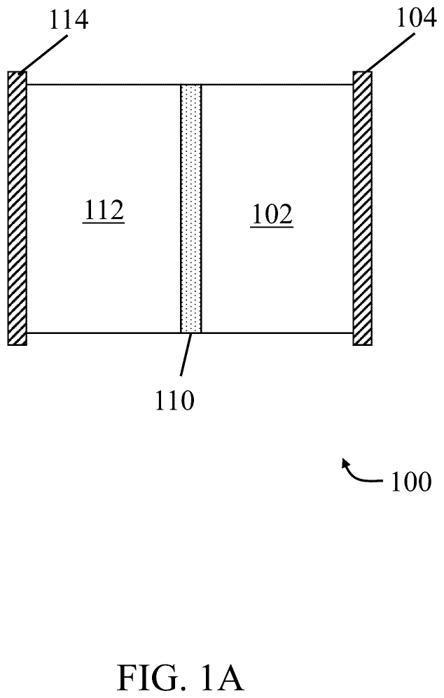

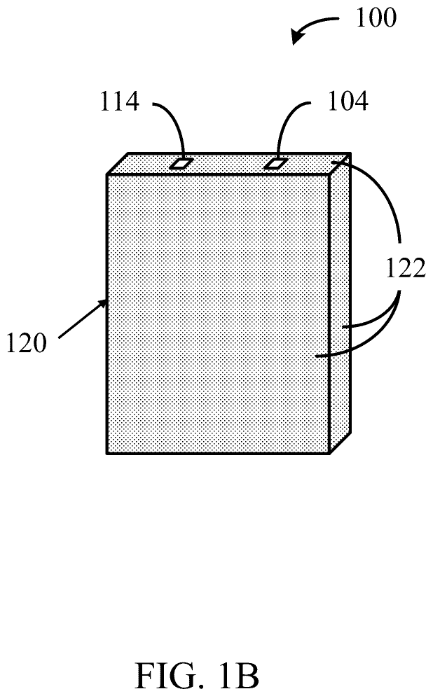

A cross section side view of an illustrative electrochemical cell 100 is shown in FIG. 1A. The electrochemical cell 100 includes a negative electrode 102 and a positive electrode 112. The electrochemical cell 100 may include a separator 110 between the negative electrode 102 and the positive electrode 112. A negative electrode current collector 104 may be electrically coupled to the negative electrode 102. A positive electrode current collector 114 may be electrically coupled to the positive electrode 112. The electrochemical cell 100 may include an electrolyte (not shown). The electrodes 102, 112 may be provided as relatively flat or planar plates or may be wrapped or wound in a spiral or other configuration (e.g., an oval configuration). Additionally or alternatively, the electrodes 102, 112 may also be provided in a folded configuration. It will be understood in light of the present disclosure that any suitable electrode configuration may be used, and the disclosure is not limited in this regard.

While discussed herein as a lithium-ion cell, it will be understood in light of the present disclosure that the electrochemical cell 100 may include any suitable chemistry. The chemistry of the electrochemical cell 100 may include, for example, lithium-metal, lithium-ion, lithium polymer, or other chemistries that may be used in or as autoclavable battery packs. In at least one embodiment, the electrochemical cell 100 includes a lithium-metal battery cell. The electrochemical cell 100 may be a primary cell or a secondary cell. In other words, the electrochemical cell 100 may or may not be rechargeable. It will be understood in light of the present disclosure that any suitable cell chemistry may be used, and the disclosure is not limited in this regard.

Generally, during charging and discharging of the electrochemical cell 100, lithium ions move between the negative electrode 102 and the positive electrode 112. For example, when the electrochemical cell 100 is discharged, lithium ions flow from the negative electrode 102 to the positive electrode 112. Conversely, when the electrochemical cell 100 is charged, lithium ions flow from the positive electrode 112 to the negative electrode 102.

An isometric view of the electrochemical cell 100 of FIG. 1A is shown in FIG. 1B. Although the electrochemical cell 100 is depicted with a prismatic shape, it will be understood in light of the present disclosure that the electrochemical cell 100 may have any suitable shape, or format, and the disclosure is not limited in this regard.

The electrochemical cell 100 may include a cell housing 120 having one or more cell exterior surfaces 122 surrounding at least a portion of each of the negative electrode 102, the positive electrode 112, the positive current collector 114, the negative current collector 104, the electrolyte, and the separator 110. A portion of each of the negative current collector 104 and the positive current collector 114 may be exposed through the one or more cell exterior surfaces 122 to allow the electrochemical cell 100 to electrically connect, or couple, to an electrical device or circuit.

The cell housing 120 may include any suitable material, such as resilient materials, or combinations of materials. Resilient (e.g., resistant to puncture and corrosion and chemically stable) material or materials may be configured to protect the internal components (e.g., the negative electrode 102, the positive electrode 112, the positive current collector 114, the negative current collector 104, the electrolyte, and the separator 110, etc.) of the electrochemical cell 100. Suitable resilient materials may include, for example, nickel, steel, titanium, aluminum, or alloys, such as alloys of nickel, steel, titanium, or aluminum. The cell housing 120 may include any suitable material or materials for holding internal components of the electrochemical cell 100 together in a predefined shape. Such packaging materials may include, for example, plastics, ceramics, etc. It will be understood in light of the present disclosure that any suitable cell housing material, or combination of materials, may be used and the disclosure is not limited in this regard.

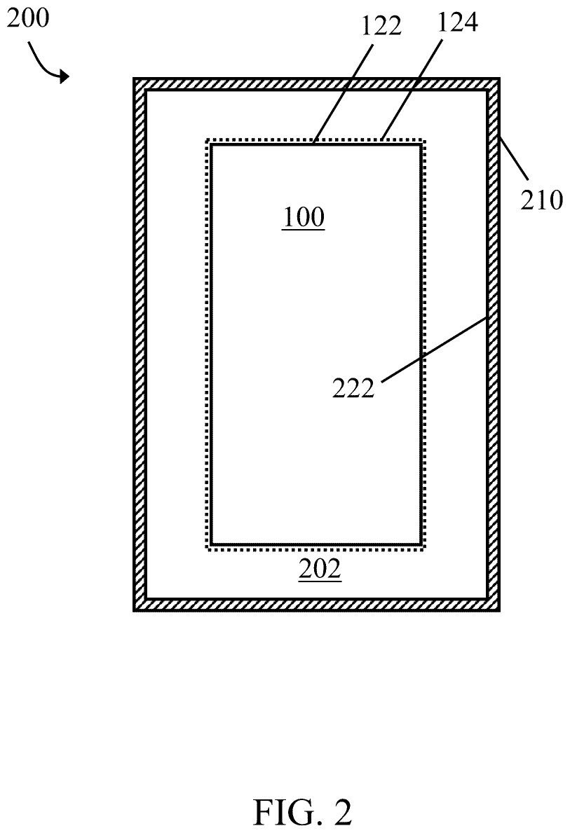

A cross-sectional representation of an illustrative battery pack 200 including the illustrative electrochemical cell 100 of FIG. 1A and having a cell thermal radiation control surface treatment is shown in FIG. 2. The battery pack 200 may include an outer case, or pack enclosure 210, including one or more pack interior surfaces 222. The pack interior surface 222 may be described as having a pack surface radiative emissivity value and a pack surface absorptive emissivity value.

The pack enclosure 210 may include any suitable materials or combinations of materials. Suitable pack enclosure 210 materials may include, for example, aluminum, titanium, stainless steel, nickel, nickel coated ferrous steels, or other alloys, such as alloys of aluminum, titanium, and nickel. In one or more embodiments, the pack enclosure 210 may include a polymeric material. It will be understood in light of the present disclosure that any suitable pack enclosure materials, or combinations of materials, may be used and the disclosure is not limited in this regard.

In one or more embodiments, and as shown in FIG. 2, the electrochemical cell 100 may be disposed within the pack enclosure 210. The electrochemical cell 100 may include a cell exterior surface 122. The cell exterior surface 122, or at least a portion of the cell exterior surface 122, may face the pack interior surface 222, or at least a portion of the pack interior surface 222. The cell exterior surface 122 may have a cell surface radiative emissivity value and a cell surface absorptive emissivity value.

The battery pack 200 may define a void, or pack-to-cell gap 202, between and separating the electrochemical cell 100 from the pack enclosure 210. More specifically, the pack-to-cell gap 202 may be defined between the cell exterior surface 122 and the pack interior surface 222. The pack-to-cell gap 202 may be, or include, an empty void, or vacuum, a partial vacuum, or may be, or include, any suitable material, or combination of materials. Suitable pack-to-cell gap materials may be selected to impede, or minimize, heat transfer via conduction and, in particular, to impede, or minimize, heat transfer via conduction from the pack enclosure 210 to the electrochemical cell 100. Suitable pack-to-cell gap materials may include air or another gas, or combinations of gases, as examples. It will be understood in light of the present disclosure that any suitable pack-to-cell gap materials, or combination of materials, may be used and the disclosure is not limited in this regard.

In at least one embodiment, the battery pack 200 may include a cell thermal radiation control surface treatment 124 on at least a portion of the cell exterior surface 122. The cell thermal radiation control surface treatment 124 may be disposed between the pack interior surface 222, or portions thereof, and the cell exterior surface 122, or portions thereof, and may be adapted to manage, or affect, transfer of thermal energy via radiation into and/or out of the electrochemical cell 100.

For example, the cell thermal radiation control surface treatment 124 may have a relatively low radiative emissivity value (e.g., a radiative emissivity value lower than the radiative emissivity value of the cell exterior surface 122) to reduce transfer of thermal energy via radiation from the electrochemical cell 100, or, more specifically, from the cell exterior surface 122. As another example, the cell surface treatment 124 may have a relatively high radiative emissivity value (e.g., a radiative emissivity value higher than the radiative emissivity value of the cell exterior surface 122) to increase transfer of thermal energy via radiation from the electrochemical cell 100, or, more specifically, from the cell exterior surface 122. As still another example, the cell surface treatment 124 may have a relatively low absorptive emissivity value (e.g., an absorptive emissivity value lower than the absorptive emissivity value of the cell exterior surface 122) to reduce transfer of thermal energy via radiation to the cell exterior surface 122 and into the electrochemical cell 100. As yet another example, the cell surface treatment 124 may have a relatively high absorptive emissivity value (e.g., an absorptive emissivity value higher than the absorptive emissivity value of the cell exterior surface 122) to increase transfer of thermal energy via radiation to the cell exterior surface 122 and into the electrochemical cell 100.

The cell thermal radiation control surface treatment 124 may have any suitable absorptive emissivity values. Suitable absorptive emissivity values of the cell surface treatment 124 may be selected based on factors such as the absorptive emissivity value of the cell exterior surface 122, (that is, the cell exterior surface 122 without a cell surface treatment 124). More specifically, suitable absorptive emissivity values of the cell surface treatment 124 may be selected to be less than the absorptive emissivity value of the cell exterior surface 122, for example, to impede heat transfer via radiation into the electrochemical cell 100 during a high-temperature autoclave operation. As another example, suitable absorptive emissivity values of the cell surface treatment 124 may be selected based on a desired radiative heat transfer, or efficiency of radiative heat transfer, into the electrochemical cell 100. For example, lower cell surface treatment absorptive emissivity values (that is, for example, lower than the cell exterior surface 122 absorptive emissivity values) may be desirable to limit, or impede, heat transfer via radiation into the electrochemical cell 100 or, more specifically, to limit, or impede, heat transfer via radiation into the electrochemical cell 100 during a high-temperature autoclave procedure, such as a steam autoclave procedure.

Suitable cell surface treatment absorptive emissivity values may include, for example, between 0 and 0.5 or between 0.1 and 0.4. As further example, cell surface treatment absorptive emissivity values may include 0.5 or less, 0.4 or less, 0.3 or less, 0.2 or less, 0.1 or less, or 0.05 or less, and/or 0 or greater, 0.05 or greater, 0.1 or greater, 0.2 or greater, 0.3 or greater, or 0.4 or greater. In one illustrative embodiment, the cell surface treatment absorptive emissivity value may be approximately 0.15. In further illustrative embodiments, the cell surface treatment absorptive emissivity value at a temperature of 100° C. may be less than the cell surface treatment absorptive emissivity value at room temperature. In still further illustrative embodiments, the cell surface treatment 124 absorptive emissivity value at room temperature may be greater than the cell exterior surface 122 absorptive emissivity value at room temperature.

The cell thermal radiation control surface treatment 124 may have any suitable radiative emissivity values. Suitable radiative emissivity values of the cell surface treatment 124 may be selected based on factors such as the radiative emissivity value of the cell exterior surface 122, (that is, the cell exterior surface 122 without a cell thermal radiation control surface treatment 124). More specifically, suitable radiative emissivity values of the cell surface treatment 124 may be selected to be greater than the radiative emissivity value of the cell exterior surface 122, for example, to improve heat transfer via radiation from the electrochemical cell 100 during typical operation. As another example, suitable radiative emissivity values of the cell surface treatment 124 may be selected based on a desired radiative heat transfer, or efficiency of radiative heat transfer, from the electrochemical cell 100. For example, higher cell surface treatment 124 radiative emissivity values (that is, for example, higher than the cell exterior surface 122 radiative emissivity values) may be desirable to increase heat transfer via radiation from the electrochemical cell 100 or, more specifically, to increase heat transfer via radiation from the electrochemical cell 100 during normal operation.

Suitable cell surface treatment radiative emissivity values may be, or include, for example, between 0.5 and 1 or between 0.9 and 1. In one illustrative embodiment, the cell surface treatment radiative emissivity value may be approximately 1. As further examples, suitable cell surface treatment radiative emissivity values may be, or include, 0.5 or greater, 0.6 or greater, 0.7 or greater, 0.8 or greater, 0.9 or greater, 0.95 or greater, or 0.99 or greater, and/or 1 or less, 0.99 or less, or 0.9 or less. In further illustrative embodiments, the cell surface treatment radiative emissivity value at a temperature of 100° C. may be less than the cell surface treatment radiative emissivity value at room temperature. In still further illustrative embodiments, the cell surface treatment 124 radiative emissivity value at room temperature may be greater than the cell exterior surface 122 radiative emissivity value at room temperature.

In one or more embodiments, the cell thermal radiation control surface treatment 124 may be, or include, a cell thermal radiation control coating. The cell surface treatment 124 may be, or include, any suitable cell surface. Suitable cell coatings may be, or include, for example, a ceramic coating, a thermochromic coating, a low absorptive emissivity coating (e.g., a coating with an absorptive emissivity value that is less than the cell surface absorptive emissivity value, 0.4 or less, 0.3 or less, 0.2 or less, 0.15 or less, or 0.1 or less), a high radiative emissivity coating (e.g., a coating with a radiative emissivity value that is greater than the cell surface radiative emissivity value, 0.6 or greater, 0.7 or greater, 0.8 or greater, or 0.9 or greater), thermal barrier coating that includes ceramic nanoparticles, a smooth coating, and/or a reflective coating. In one embodiment, the cell thermal radiation control surface treatment 124 may include a cell coating with both low absorptive emissivity and high radiative emissivity. Suitable coatings with both low absorptive emissivity and high radiative emissivity may include, for example, low-E paint, dual coating layers (e.g., a bottom/surface layer with low absorptive emissivity covered by a second layer with high radiative emissivity), and/or SUPER THERM CERAMIC COATING (available from NEOTECH COATINGS AUSTRALIA of Stepney, South Australia). As another example, suitable coatings with both low absorptive emissivity and high radiative emissivity may be, or include, sputtering compounds, such as metallic coatings (e.g., silver and/or gold). As yet another example, suitable coatings with both low absorptive emissivity and high radiative emissivity may be, or include, pyrolytic compounds, such as fluorine-doped tin oxide (FTO). In some embodiments, deposition techniques may be used to promote thin-film microstructure and to enhance thin-film properties may be used, such as a seed layer. For example, a zinc oxide (ZnO) seed layer may improve silver thin film crystallite size and/or grain size, which may reduce resistivity and absorption. Without wishing to be bound by theory, a cell coating with both low absorptive emissivity and high radiative emissivity may be desirable to both impede radiative heat transfer into the electrochemical cell 100 and enhance radiative heat transfer from the electrochemical cell 100.

In at least one embodiment, the cell thermal radiation control surface treatment 124 may be, or include, a modification to the cell exterior surface 122 to increase or decrease the emissivity of the cell exterior surface 122. For example, the cell surface treatment 124 may be, or include, smoothed, polished, or reflective surface of the cell exterior surface 122. Without wishing to be bound by theory, emissivity of a surface may generally decrease as the smoothness or reflectiveness of the surface increases.

In some embodiments, the cell thermal radiation control surface treatment 124 may be, or include, a thermochromic coating. A thermochromic coating may be described as a coating including a thermochromic compound, or a combination of thermochromic compounds, such as a thermochromic pigment or dye. Suitable thermochromic coatings may be selected based on factors such as relative emissivity values at room temperature and elevated temperatures (such as temperatures of 100° C. or greater). For example, a suitable thermochromic coating may have an emissivity value, or values, at room temperature higher than the coating's emissivity value at a temperature of 100° C. or greater. As another example, a suitable thermochromic coating may have an emissivity value, or values, at an elevated temperature (e.g., a temperature of 100° C. or greater) that is less than an emissivity value of the cell exterior surface at the elevated temperature and the suitable thermochromic compound may have an emissivity value, or values, at room temperature that is greater than the thermochromic compound emissivity value, or values, at the elevated temperature. The thermochromic coating may include any suitable thermochromic compound or combination of thermochromic compounds, such as leuco dyes or liquid crystal. Thermochromic materials are generally organic leuco-dye mixtures, which include a color-former, a color-developer, and a solvent. The color-former may be described as determining the base color and may be, for example, a cyclic ester. The color developer may be described as producing the color change and the final color intensity and may be, for example, a weak acid. The melting point of the solvent determines the color transition temperature and may be, for example, an alcohol or an ester. It will be understood in light of the present disclosure that any suitable thermochromic compounds may be used and the disclosure is not limited in this regard. Furthermore, suitable thermochromic compounds may be selected based on factors, such as those described herein.

In some embodiments, the cell thermal radiation control surface treatment 124 may be adapted to have the suitable emissivity values described herein at an elevated surface temperature, such as when a temperature of the cell exterior surface 122 and/or a temperature of the cell thermal radiation control surface treatment 124 is elevated. Cell surface temperatures may be elevated, for example, during a high-temperature autoclave procedure. As an example, the cell surface treatment 124 may have any of the suitable emissivity values (e.g., the suitable cell surface treatment absorptive emissivity values and the suitable cell surface treatment radiative emissivity values) described herein at a temperature of 90° C. or greater, 100° C. or greater, 110° C. or greater, 120° C. or greater, or 140° C. or greater. In one embodiment, the cell surface treatment 124 may have a cell surface treatment absorptive emissivity value at a temperature of 100° C. or greater that is less than the cell exterior surface 122 absorptive emissivity value at the temperature of 100° C.

In at least one embodiment, the cell thermal radiation control surface treatment 124 may be adapted to have temperature-dependent emissivity values. For example, the cell surface treatment 124 may have a cell surface treatment emissivity value at a temperature of 100° C. or greater that is less than the cell surface treatment emissivity value at room temperature. For example, and as described herein, the cell thermal radiation control surface treatment 124 may be, or include, a thermochromic coating. A cell surface treatment emissivity value that is lower at an elevated temperature compared with room temperature may be desirable, for example, to reduce thermal energy transfer via radiation into the electrochemical cell 100 while the cell is in a high-temperature environment and, further, to increase thermal energy transfer via radiation from the electrochemical cell 100 at typical operating temperatures, such as at ambient room temperature. In other words, a lower cell surface treatment emissivity value at elevated temperatures may be desirable to impede, or mitigate, heat transfer via radiation from outside the battery pack 200, through the pack enclosure 210, and into the cell 100 during a high-temperature autoclave operation, while also allowing heat transfer via radiation from the cell 100 in typical operating conditions, such as in ambient room temperature.

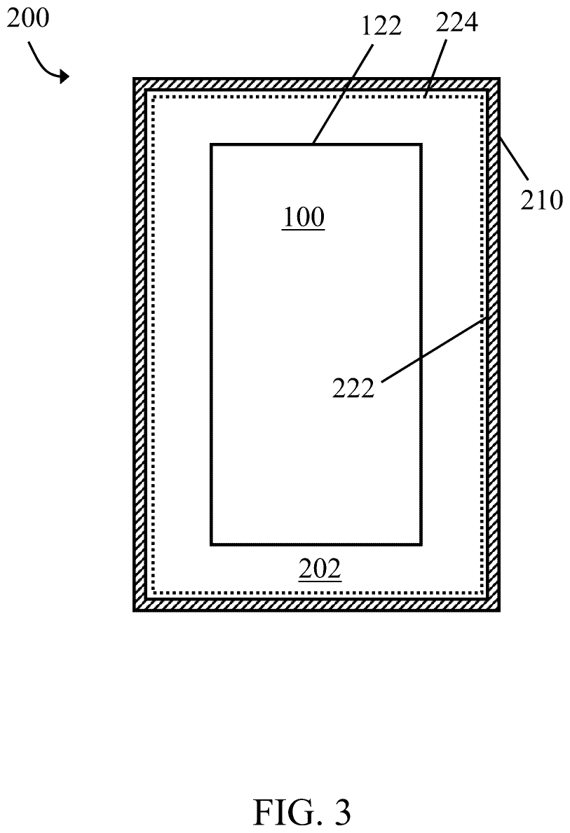

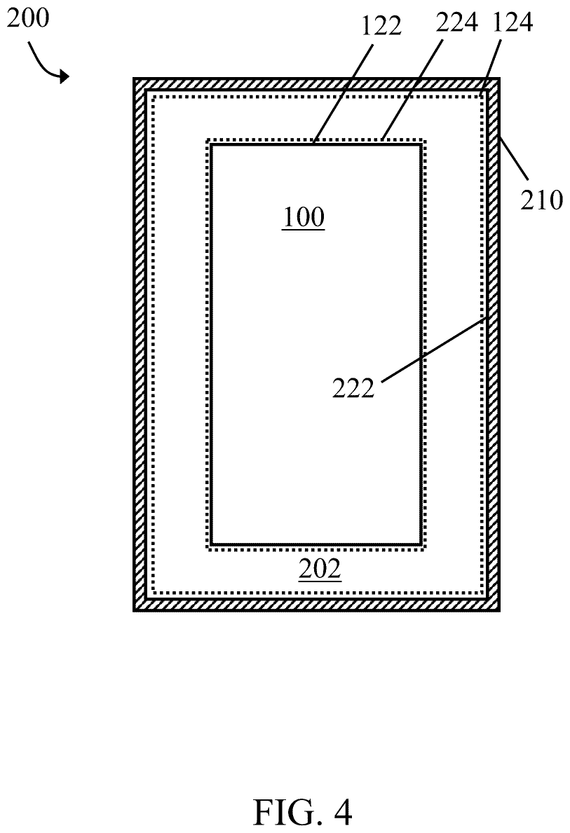

In one or more embodiments, the battery pack 200 may include a pack thermal radiation control surface treatment 224 on at least a portion of the pack interior surface 222. A cross-sectional representation of another illustrative battery pack 200 including the illustrative electrochemical cell 100 of FIG. 1A and having a pack thermal radiation control surface treatment 224 is shown in FIG. 3. In some embodiments, the battery pack 200 may include the pack thermal radiation control surface treatment 224 on at least a portion of the pack interior surface 222 and the cell thermal radiation control surface treatment 124 on at least a portion of the cell exterior surface 122. A cross-sectional representation of yet another illustrative battery pack 200 including the illustrative electrochemical cell 100 of FIG. 1A and having both of a cell thermal radiation control surface treatment 124 and a pack thermal radiation control surface treatment 224 is shown in FIG. 4. The pack surface treatment 224 may be disposed between the pack interior surface 222 and the cell exterior surface 122 and may be adapted to help manage, or affect, radiative transfer of thermal energy into and/or out of the electrochemical cell 100. More specifically, the pack surface treatment 224 may be adapted to impede, or mitigate, radiative heat transfer from the pack enclosure 210 through the pack-to-cell gap 202 and into the electrochemical cell 100 and/or to enhance, or increase, radiative heat transfer into (i.e., absorption into) the pack enclosure 210 from the electrochemical cell 100.

For example, the pack thermal radiation control surface treatment 224 may have a relatively low radiative emissivity value (e.g., a radiative emissivity value lower than the radiative emissivity value of the pack interior surface 222) to reduce transfer of thermal energy via radiation from the pack enclosure 210, or from the pack interior surface 222. As another example, the pack surface treatment 224 may have a relatively high radiative emissivity value (e.g., a radiative emissivity value higher than the radiative emissivity value of the pack interior surface 222) to increase transfer of thermal energy via radiation from the pack enclosure 210, or from the pack interior surface 222, which may be desirable, for example, to distribute thermal energy through the pack enclosure 210 by radiating thermal energy from the pack interior surface 222 and re-absorb the thermal energy returned, or reflected, from the electrochemical cell 100, or the cell exterior surface 122. As yet another example, the pack surface treatment 224 may have a relatively high absorptive emissivity value (e.g., an absorptive emissivity value higher than the absorptive emissivity value of the pack interior surface 222) to increase transfer of thermal energy via radiation to the pack interior surface 222, or into the pack enclosure 210, and, more specifically, to increase absorption by the pack enclosure 210 of thermal energy radiated from the cell exterior surface 122, or the cell thermal radiation control surface treatment 124.

The pack thermal radiation control surface treatment 224 may have any suitable absorptive emissivity values. Suitable absorptive emissivity values of the pack surface treatment 224 may be selected based on factors such as the absorptive emissivity value of the pack interior surface 222, (that is, the pack interior surface 222 without a pack surface treatment 224). More specifically, suitable absorptive emissivity values of the pack surface treatment 224 may be selected to be greater than the absorptive emissivity value of the pack interior surface 222, for example, to enhance, or increase, heat transfer via radiation into the pack enclosure 210 (i.e., thermal energy absorbed by the pack interior surface 222) from the electrochemical cell 100, such as during a high-temperature autoclave operation.

Suitable pack surface treatment absorptive emissivity values may be, or include, for example, between 0.5 and 1 or between 0.9 and 1. In one illustrative embodiment, the pack surface treatment absorptive emissivity value may be approximately 1. As further examples, suitable pack surface treatment absorptive emissivity values may be, or include, 0.5 or greater, 0.6 or greater, 0.7 or greater, 0.8 or greater, 0.9 or greater, 0.95 or greater, or 0.99 or greater, and/or 1 or less, 0.99 or less, or 0.9 or less. In further illustrative embodiments, the pack surface treatment absorptive emissivity value at an elevated temperature (e.g., a temperature of 100° C. or greater) may be greater than the absorptive emissivity value of the pack interior surface 222 at the elevated temperature.

In one or more embodiments, the pack thermal radiation control surface treatment 224 may include a pack thermal radiation control coating. The pack surface treatment 124 may be, or include, any suitable pack coating. Suitable pack coatings may be, or include, for example, a thermochromic coating, a high absorptive emissivity coating (e.g., an absorptive emissivity value that is greater than the pack surface absorptive emissivity value, 0.6 or greater, 0.7 or greater, 0.8 or greater, or 0.9 or greater), a high radiative emissivity coating (e.g., a radiative emissivity value that is greater than the pack surface radiative emissivity value, 0.6 or greater, 0.7 or greater, 0.8 or greater, or 0.9 or greater), and/or a rough coating. In one embodiment, the pack thermal radiation control surface treatment 224 may be, or include, a pack coating with both high absorptive emissivity and high radiative emissivity. Without wishing to be bound by theory, a pack coating with both high absorptive emissivity and high radiative emissivity may be desirable to enhance absorption into the pack enclosure 210 of thermal energy radiated from the electrochemical cell 100 and to spread the absorbed thermal energy throughout the material of the pack enclosure 210.

In at least one embodiment, the pack thermal radiation control surface treatment 224 may be, or include, a modification to the pack interior surface 222 to increase or decrease the emissivity of the pack interior surface 222. For example, the pack surface treatment 224 may be, or include, a roughened or textured surface. Without wishing to be bound by theory, emissivity of a surface may generally increase with increased surface roughness. The pack surface treatment 224 that is, or includes, a roughened or textured surface may be desirable, for example, to increase radiative emissivity of the pack interior surface 222, increase absorptive emissivity of the pack interior surface 222, or both.

In some embodiments, the pack thermal radiation control surface treatment 224 may be adapted to have the suitable emissivity values described herein at an elevated surface temperature, such as when a temperature of the pack interior surface 222 and/or a temperature of the pack surface treatment 224 is elevated. Pack surface temperatures may be elevated, for example, during a high-temperature autoclave procedure. As an example, the pack surface treatment 224 may have any of the suitable emissivity values (e.g., the suitable pack surface treatment absorptive emissivity values and the suitable pack surface treatment radiative emissivity values) described herein at a temperature of 90° C. or greater, 100° C. or greater, 110° C. or greater, 120° C. or greater, or 140° C. or greater. In one embodiment, the pack surface treatment 224 may have a pack surface treatment absorptive emissivity value at a temperature of 100° C. or greater that is greater than the pack interior surface 222 absorptive emissivity value at the temperature of 100° C.

In at least one embodiment, the illustrative battery pack 200 may include the cell surface treatment 124 with a low absorptive emissivity (e.g., an absorptive emissivity value that is less than the cell surface absorptive emissivity value, 0.4 or less, 0.3 or less, 0.2 or less, 0.15 or less, or 0.1 or less) and a high radiative emissivity (e.g., a radiative emissivity value that is greater than the cell surface radiative emissivity value, 0.6 or greater, 0.7 or greater, 0.8 or greater, or 0.9 or greater) and a pack surface treatment 224 with high absorptive emissivity and high radiative emissivity.

In some embodiments, the illustrative battery pack 200 may include the cell surface treatment 124 including a thermochromic coating configured to be dark, or have high emissivity (e.g., emissivity values that are greater than the cell surface emissivity values, 0.6 or greater, 0.7 or greater, 0.8 or greater, or 0.9 or greater), at typical operating temperatures (e.g., room temperature) and to be light, or have low emissivity (e.g., emissivity values that are less than the cell surface emissivity values, 0.4 or less, 0.3 or less, 0.2 or less, 0.15 or less, or 0.1 or less) at elevated temperatures (e.g., 100° C. or greater, such as during a high-temperature autoclave procedure) and a pack surface treatment 224 with low radiative emissivity.

In one or more embodiments, the illustrative battery pack 200 may include the cell surface treatment 124 with a smooth, reflective surface and a pack surface treatment 224 with a high surface roughness or a textured surface.

While the illustrative battery packs 200 is generally described herein as having one electrochemical cell (i.e., the electrochemical cell 100) disposed therein, it will be understood in light of the present disclosure that battery packs including any number of electrochemical cells disposed therein are contemplated, and the disclosure is not limited in this regard.

### Illustrative Aspects

The following is a list of illustrative aspects according to the present disclosure.

Aspect 1 is a battery pack comprising: a pack enclosure comprising a pack interior surface, the pack interior surface having a pack surface radiative emissivity value and a pack surface absorptive emissivity value; an electrochemical cell disposed within the pack enclosure, the electrochemical cell comprising a cell exterior surface facing the pack interior surface, the cell exterior surface having a cell surface radiative emissivity value and a cell surface absorptive emissivity value; and a cell thermal radiation control surface treatment on at least a portion of the cell exterior surface and disposed between the pack interior surface and the cell exterior surface, the cell surface treatment having a cell surface treatment absorptive emissivity value at a temperature of 100° C. or greater that is less than the cell surface absorptive emissivity value at the temperature.

Aspect 2 is the battery pack of aspect 1, wherein the cell thermal radiation control surface treatment comprises a thermochromic compound.

Aspect 3 is the battery pack of any one of aspects 1-2, wherein the cell surface treatment absorptive emissivity value at the temperature is less than the cell surface treatment absorptive emissivity value at room temperature, wherein the cell surface treatment radiative emissivity value at the temperature is greater than the cell surface treatment radiative emissivity value at room temperature, or both.

Aspect 4 is the battery pack of any one of aspects 2-3, wherein the cell surface treatment absorptive emissivity value at room temperature is greater than the cell surface absorptive emissivity value at room temperature; wherein the cell surface treatment radiative emissivity value at room temperature is greater than the cell surface radiative emissivity value at room temperature; or both.

Aspect 5 is the battery pack of any one of aspects 1-4, wherein the cell surface treatment absorptive emissivity value at the temperature is 0.4 or less, 0.3 or less, 0.2 or less, 0.1 or less, or between 0 and 0.5.

Aspect 6 is the battery pack of any one of aspects 1-5, wherein the cell surface treatment radiative emissivity value at the temperature is 0.6 or greater, 0.7 or greater, 0.8 or greater, 0.9 or greater, or between 0.6 and 1.

Aspect 7 is the battery pack of any one of aspects 1-6, further comprising a pack thermal radiation control surface treatment on at least a portion of the pack interior surface and disposed between the pack interior surface and the cell exterior surface, the pack thermal radiation surface treatment having:
a pack surface treatment absorptive emissivity value greater than the pack surface absorptive emissivity value; a pack surface treatment radiative emissivity value greater than the pack surface radiative emissivity value; or both.

Aspect 8 is the battery pack of any one of aspects 1-7, further comprising a pack thermal radiation control surface treatment on at least a portion of the pack interior surface and disposed between the pack interior surface and the cell exterior surface, the pack thermal radiation control surface treatment having:
a pack surface treatment absorptive emissivity value less than the pack surface absorptive emissivity value; a pack surface treatment radiative emissivity value less than the pack surface radiative emissivity value; or both.

Aspect 9 is the battery pack of any one of aspects 7-8, wherein the pack thermal radiation control surface treatment comprises a surface roughness to increase radiative emissivity of the pack interior surface, increase absorptive emissivity of the pack interior surface, or both.

Aspect 10 is the battery pack of any one of aspects 1-9, wherein the cell thermal radiation control surface treatment comprises a reflective surface.

Aspect 11 is a battery pack comprising:
a pack enclosure comprising a pack interior surface, the pack interior surface having a pack surface radiative emissivity value and a pack surface absorptive emissivity value;
an electrochemical cell disposed within the pack enclosure, the electrochemical cell comprising a cell exterior surface facing the pack interior surface, the cell exterior surface having a cell surface radiative emissivity value and a cell surface absorptive emissivity value; and
a pack thermal radiation control surface treatment on at least a portion of the pack interior surface and disposed between the pack interior surface and the cell exterior surface, the pack surface treatment having a pack surface treatment absorptive emissivity value at a temperature of 100° C. or greater that is greater than the pack surface absorptive emissivity value at the temperature, a pack surface treatment radiative emissivity value at the temperature that is greater than the pack surface radiative emissivity value at the temperature, or both.

Aspect 12 is the battery pack of aspect 11, wherein the pack surface treatment absorptive emissivity value at room temperature is greater than the pack surface absorptive emissivity value at room temperature; wherein the pack surface treatment radiative emissivity value at room temperature is greater than the pack surface radiative emissivity value at room temperature; or both.

Aspect 13 is the battery pack of any one of aspects 11-12, wherein the pack surface treatment absorptive emissivity value at the temperature is 0.6 or greater, 0.7 or greater, 0.8 or greater, 0.9 or greater, or between 0.6 and 1.

Aspect 14 is the battery pack of any one of aspects 11-13, wherein the pack surface treatment radiative emissivity value at the temperature is 0.6 or greater, 0.7 or greater, 0.8 or greater, 0.9 or greater, or between 0.6 and 1.

Aspect 15 is the battery pack of any one of aspects 11-14, further comprising a cell thermal radiation control surface treatment on at least a portion of the cell exterior surface and disposed between the pack interior surface and the cell exterior surface, the cell thermal radiation control surface treatment having: a cell surface treatment absorptive emissivity value less than the cell surface absorptive emissivity value; a cell surface treatment radiative emissivity value greater than the cell surface radiative emissivity value; or both.

Aspect 16 is a battery pack comprising:
a pack enclosure comprising a pack interior surface;
an electrochemical cell disposed within the pack enclosure, the electrochemical cell comprising a cell exterior surface facing the pack interior surface; and
a cell thermal radiation control surface treatment on at least a portion of the cell exterior surface and disposed between the pack interior surface and the cell exterior surface, the cell thermal radiation control surface treatment having a cell coating absorptive emissivity value of 0.4 or less, 0.3 or less, 0.2 or less, or 0.1 or less at a temperature of 100° C. or greater.

Aspect 17 is the battery pack of aspect 16, wherein the cell thermal radiation control surface treatment comprises a thermochromic compound.

Aspect 18 is the battery pack of any one of aspects 16-17, wherein the cell surface treatment absorptive emissivity value at the temperature of 100° C. or greater is less than the cell surface treatment absorptive emissivity value at room temperature, wherein the cell surface treatment radiative emissivity value at the temperature is greater than the cell surface treatment radiative emissivity value at room temperature, or both.

Aspect 19 is the battery pack of any one of aspects 16-18, wherein the cell surface treatment absorptive emissivity value at room temperature is greater than the cell surface absorptive emissivity value at room temperature; wherein the cell surface treatment radiative emissivity value at room temperature is greater than the cell surface radiative emissivity value at room temperature; or both.

Aspect 20 is the battery pack of any one of aspects 16-19, wherein the cell surface treatment absorptive emissivity value at the temperature of 100° C. or greater is less than the cell surface absorptive emissivity value at the temperature, wherein the cell surface treatment radiative emissivity value at the temperature is greater than the cell surface radiative emissivity at the temperature, or both.

It should also be noted that, as used in this specification and the appended claims, the phrase “configured” describes a system, apparatus, or other structure that is constructed to perform a particular task or adopt a particular configuration. The word “configured” can be used interchangeably with similar words such as “arranged,” “constructed,” “manufactured,” and the like.

It should further be noted that, as used in this specification and the appended claims, reference to numbers of electrodes is merely for the purpose of distinguishing between electrodes and does not necessarily limit the number of electrodes.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

What is claimed is:

1. A battery pack comprising:
a pack enclosure comprising a pack interior surface, the pack interior surface having a pack surface radiative emissivity value and a pack surface absorptive emissivity value;
an electrochemical cell disposed within the pack enclosure, the electrochemical cell comprising a cell exterior surface facing the pack interior surface, the cell exterior surface having a cell surface radiative emissivity value and a cell surface absorptive emissivity value; and
a cell thermal radiation control surface treatment on at least a portion of the cell exterior surface and disposed between the pack interior surface and the cell exterior surface, the cell surface treatment having a cell surface treatment absorptive emissivity value at a temperature of 100° C. or greater that is less than the cell surface absorptive emissivity value at the temperature.

2. The battery pack of claim 1, wherein the cell thermal radiation control surface treatment comprises a thermochromic compound.

3. The battery pack of claim 1, wherein the cell surface treatment absorptive emissivity value at the temperature is less than the cell surface treatment absorptive emissivity value at room temperature, wherein the cell surface treatment radiative emissivity value at the temperature is greater than the cell surface treatment radiative emissivity value at room temperature, or both.

4. The battery pack of claim 1, wherein the cell surface treatment absorptive emissivity value at room temperature is greater than the cell surface absorptive emissivity value at room temperature; wherein the cell surface treatment radiative emissivity value at room temperature is greater than the cell surface radiative emissivity value at room temperature; or both.

5. The battery pack of claim 1, wherein the cell surface treatment absorptive emissivity value at the temperature is 0.4 or less.

6. The battery pack of claim 1, wherein the cell surface treatment radiative emissivity value at the temperature is 0.6 or greater.

7. The battery pack of claim 1, further comprising a pack thermal radiation control surface treatment on at least a portion of the pack interior surface and disposed between the pack interior surface and the cell exterior surface, the pack thermal radiation surface treatment having:
a pack surface treatment absorptive emissivity value greater than the pack surface absorptive emissivity value;
a pack surface treatment radiative emissivity value greater than the pack surface radiative emissivity value; or
both.

8. The battery pack of claim 1, further comprising a pack thermal radiation control surface treatment on at least a portion of the pack interior surface and disposed between the pack interior surface and the cell exterior surface, the pack thermal radiation control surface treatment having:
a pack surface treatment absorptive emissivity value less than the pack surface absorptive emissivity value;
a pack surface treatment radiative emissivity value less than the pack surface radiative emissivity value; or
both.

9. The battery pack of claim 1, wherein the pack thermal radiation control surface treatment comprises a surface roughness to increase radiative emissivity of the pack interior surface, increase absorptive emissivity of the pack interior surface, or both.

10. The battery pack of claim 1, wherein the cell thermal radiation control surface treatment comprises a reflective surface.

11. A battery pack comprising:
a pack enclosure comprising a pack interior surface, the pack interior surface having a pack surface radiative emissivity value and a pack surface absorptive emissivity value;
an electrochemical cell disposed within the pack enclosure, the electrochemical cell comprising a cell exterior surface facing the pack interior surface, the cell exterior surface having a cell surface radiative emissivity value and a cell surface absorptive emissivity value; and
a pack thermal radiation control surface treatment on at least a portion of the pack interior surface and disposed between the pack interior surface and the cell exterior surface, the pack surface treatment having a pack surface treatment absorptive emissivity value at a temperature of 100° C. or greater that is greater than the pack surface absorptive emissivity value at the temperature, a pack surface treatment radiative emissivity value at the temperature that is greater than the pack surface radiative emissivity value at the temperature, or both.

12. The battery pack of claim 11, wherein the pack surface treatment absorptive emissivity value at room temperature is greater than the pack surface absorptive emissivity value at room temperature; wherein the pack surface treatment radiative emissivity value at room temperature is greater than the pack surface radiative emissivity value at room temperature; or both.

13. The battery pack of claim 11, wherein the pack surface treatment absorptive emissivity value at the temperature is 0.4 or less.

14. The battery pack of claim 11, wherein the pack surface treatment radiative emissivity value at the temperature is between 0.6 and 1.

15. The battery pack of claim 11, further comprising a cell thermal radiation control surface treatment on at least a portion of the cell exterior surface and disposed between the pack interior surface and the cell exterior surface, the cell thermal radiation control surface treatment having:
a cell surface treatment absorptive emissivity value less than the cell surface absorptive emissivity value;
a cell surface treatment radiative emissivity value greater than the cell surface radiative emissivity value; or
both.

16. A battery pack comprising:
a pack enclosure comprising a pack interior surface;
an electrochemical cell disposed within the pack enclosure, the electrochemical cell comprising a cell exterior surface facing the pack interior surface; and
a cell thermal radiation control surface treatment on at least a portion of the cell exterior surface and disposed between the pack interior surface and the cell exterior surface, the cell thermal radiation control surface treatment having a cell coating absorptive emissivity value of 0.4 or less, 0.3 or less, 0.2 or less, or 0.1 or less at a temperature of 100° C. or greater.

17. The battery pack of claim 16, wherein the cell thermal radiation control surface treatment comprises a thermochromic compound.

18. The battery pack of claim 16, wherein the cell surface treatment absorptive emissivity value at the temperature of 100° C. or greater is less than the cell surface treatment absorptive emissivity value at room temperature, wherein the cell surface treatment radiative emissivity value at the temperature is greater than the cell surface treatment radiative emissivity value at room temperature, or both.

19. The battery pack of claim 16, wherein the cell surface treatment absorptive emissivity value at room temperature is greater than the cell surface absorptive emissivity value at room temperature; wherein the cell surface treatment radiative emissivity value at room temperature is greater than the cell surface radiative emissivity value at room temperature; or both.

20. The battery pack of claim 16, wherein the cell surface treatment absorptive emissivity value at the temperature of 100° C. or greater is less than the cell surface absorptive emissivity value at the temperature, wherein the cell surface treatment radiative emissivity value at the temperature is greater than the cell surface radiative emissivity at the temperature, or both.
